(19) United States
(12) Patent Application Publication
(10) Pub. No.: US 2012/0041453 A1
(43) Pub. Date: Feb. 16, 2012

(54) Fastening Device for a Mitral Valve and Method

(76) Inventor: Klaus Klingenbeck, Aufsess (DE)

(21) Appl. No.: 13/208,462

(22) Filed: Aug. 12, 2011

(30) Foreign Application Priority Data
Aug. 13, 2010 (DE) 10 2010 039 304.5

(51) Int. Cl. A61B 17/00

(52) U.S. Cl. 606/139; 606/151

(57) ABSTRACT

A fastening device connecting two flaps of a mitral valve of a human heart is provided. The fastening device has a pair of fixing arms each with a connection side. The fixing arms are connected to each other at a first end by a joint and each have a second free end. The fixing arms are able to be moved from an opened position, in which the connecting sides enclose an angle of at least 5° to each other, into the closed position in which the connecting sides rest essentially in parallel on one another. The fastening device has grip elements, which together with the fixing arms, at least partly connect the two flaps of the mitral valve in the closed position of the fixing arms. The fastening device has a position detection system which measures the spatial position and the orientation of the fastening device.

### CROSS REFERENCE TO RELATED APPLICATIONS

This application claims priority of German application No. 10 2010 039 304.5 filed Aug. 13, 2010, which is incorporated by reference herein in its entirety.

### FIELD OF THE INVENTION

The invention relates to a fastening device for connecting two flaps of a mitral valve of the human heart and also to a method for delivering a fastening device.

### BACKGROUND OF THE INVENTION

Minimally-invasive procedures are increasingly being carried out for repairing heart valves. A mitral regurgitation is a malfunction of the mitral valve, the connecting valve between left atrium and left ventricle, which causes the mitral valve to leak and not to close correctly, resulting in a flow of blood back into the left atrium and thereby to reduced blood flow, resulting in many consequential problems, including heart failure. A mitral regurgitation can for example be treated by means of a procedure in which a fastening device for the mitral valve, known as the MitraClip™ is delivered by means of a catheter and a delivery system into the heart and is positioned there so that the MitraClip™ remains in the heart and permanently holds together the two flaps of the mitral valve at the leakage point, known for example from patent WO 2004/103162 A2. High-quality, 3-dimensional imaging of the anatomy of the heart is of increasing importance for the planning and execution of the procedure. Various modalities are available for the imaging, which can be used both pre-interventionally and also during the intervention: MSCT, Cardiac MRI, 3D-US, Cardiac DynaCT, etc. Corresponding 3D volume images are used as roadmaps for the navigation of the MitraClip™.

### SUMMARY OF THE INVENTION

The object of the present invention is to provide a device which makes possible improved delivery of a fastening device for the mitral valve. A further object of the invention is to provide a method for delivering such a fastening device for the mitral valve.

The object is inventively achieved by a fastening device for a mitral valve and a method for introducing the fastening device in accordance with the independent claims. Advantageous embodiments of the invention are the subject matter of the associated dependent claims in each case.

The inventive fastening device for connecting two flaps of a mitral valve of the human heart having a pair of fixing arms each with a connection side, which fixing arms are connected to each other at a first end by means of a joint and have a second, free end in each case, whereby they are able to be moved from an opened position in which the connection sides enclose an angle of at least 5° to each other, into a closed position in which the connection sides are substantially parallel to each other, and having grip elements which, together with the fixing arms, in the closed position of the fixing arms, at least partly connect the two flaps of the mitral valve, is assigned a position detection system which is embodied to measure the spatial position and the orientation of the fastening device. To this end the position detection system has a position sensor to detect measurement data for determining the spatial position and orientation, the position detection system can also have an evaluation device for evaluating the measurement data of the position sensor. The fastening device can be positioned and delivered by means of the invention without errors and thus in an especially easy and uncomplicated manner. It is no longer necessary for a user to rely solely on visual data in order to carry out the positioning, but rather an automatic, secure and precise delivery can be guaranteed. What is known as a MitraClip™ can be used as the fastening device for example.

In accordance with an embodiment of the invention the fastening device has a delivery system which is arranged detachably on the fastening device. Such a delivery system is for example also described in publication WO 2004/103162 A2. It can be attached to the fastening device by means the mechanism and after correct arrangement of the fastening device in the mitral valve can be removed together with a catheter on which it is inserted into the body. The catheter can be moved manually or for example by means of magnetic navigation in the body of the patient.

In accordance with a further embodiment of the invention the position sensor is arranged on an articulated joint or on a fixing arm. In this way it does not adversely affect the function of the grip arms of the fastening device and can be used after completion of the positioning for further checking of the correct position of the fastening device.

Advantageously the position sensor is arranged on the delivery system. In this way the position sensor can be removed from the heart of the patient together with the delivery system after completion of the positioning.

In accordance with a further embodiment of the invention the position sensor is formed by at least one RFID transponder. A known and proven technology is involved here, so that equipping the fastening device with one or more RFIDs is especially easy and effortless. The position detection system in this case has at least one read facility for reading out the position information of the RFID transponder, whereby the information is read out by short-range magnetic alternating fields or by high-frequency radio waves.

In accordance with a further embodiment of the invention the position sensor has a least one field coil, especially a number of crossed coils, e.g. miniature coils. Such an electromagnetic position detection system forms a reliable option for determining positions and orientations of objects in space.

In accordance with an embodiment of the invention the position detection system and the position sensor are embodied to jointly determine three space coordinates as well as at least two, especially three, direction angles of the fastening device. The space coordinates can for example define the precise position of the fastening device in space relative to Cartesian coordinates and in relation to a coordinate source. At least two more direction angles are necessary for orientation since the fastening device involves an extended body (by contrast to an extensionless point). The position sensor can also be referred to for this reason as a 5D or 6D position sensor.

Advantageously, for additional use of magnetic navigation, the fastening device also has a least one magnetic dipole element. In this way, by means of a system for magnetic navigation, as is offered by the Stereotaxis company for example, positioning and delivery of the fastening device or of the delivery system can be supported. This makes further checking and improvement of the introduction and positioning of the fastening device possible, so that the minimally-invasive intervention can be carried out even more quickly and safely.

A delivery of the fastening device with position sensor into the mitral valve can be monitored for example by means of an X-ray system having a recording system for recording 3D volume datasets of an object under examination and also a position detection system.

An inventive method for delivering the fastening device into the mitral valve of a human heart has the following steps:
Creation of a 3D volume dataset of the region surrounding the mitral valve,
Segmentation of the mitral valve,
Determination of a mitral valve plane of the mitral valve,
Selection of a target position and target orientation for the fastening device,
Regulating the position of the fastening device using the data of the position sensor until such time as the position of the fastening device matches the target position, and
Regulating the orientation of the fastening device using the data of the position sensor, until such time as the orientation of the fastening device matches the target position.

### BRIEF DESCRIPTION OF THE DRAWINGS

The invention as well as further advantageous embodiments in accordance with features of the dependent claims will be explained below in greater detail with reference to schematically represented exemplary embodiments in the drawing, without the invention being restricted to these exemplary embodiments. The figures show:

FIG. 1 a view of the anatomy of a human heart,

FIG. 2 a view of a hinged-open inventive fastening device with a position sensor,

FIG. 3 a view from above of an opening in a mitral valve with the fastening device correctly inserted,

FIG. 4 a view of a closed inventive fastening device with a position sensor,

FIG. 5 an overhead view of a mitral valve plane,

FIG. 6 a perspective view of the mitral valve plane and the associated normal vector,

FIG. 7 a perspective view of the mitral valve plane and the target position of the fastening device, and

FIG. 8 a flow diagram of the inventive method.

### DETAILED DESCRIPTION OF THE INVENTION

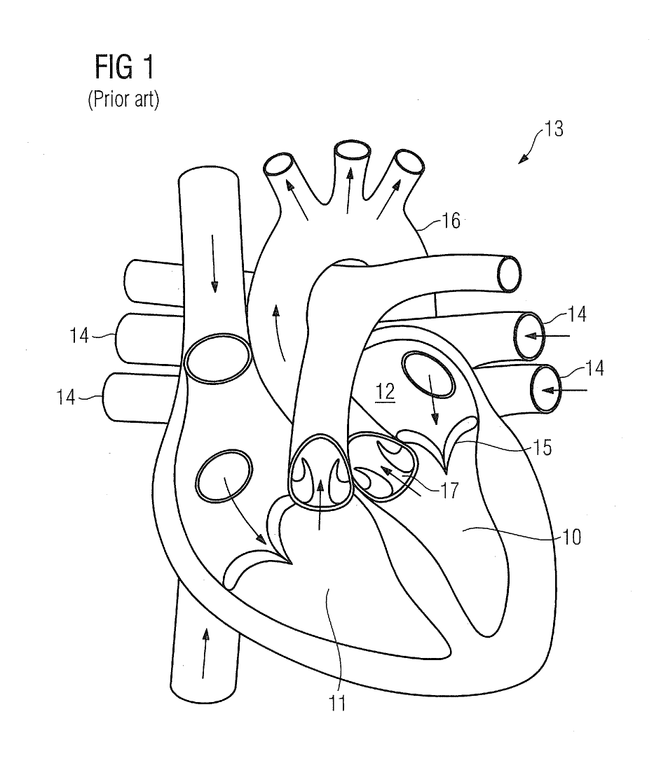

A human heart 13 is shown in FIG. 1, with only the parts of relevance for the invention being described. The heart 13 has a left ventricle 10, a right ventricle 11 and a left atrium 12. Blood enriched with oxygen flows into the left atrium 12 through the lung veins 14, from there it flows via the mitral valve 15 into the left ventricle 10 and via the aorta valve 17 and the aorta 16 into the blood circulation. A problem with the ability of the mitral valve to close results in a flowback of blood from the left ventricle into the left atrium. In order to reduce this problem a fastening device, known as a MitraClip™, can be implanted into the heart.

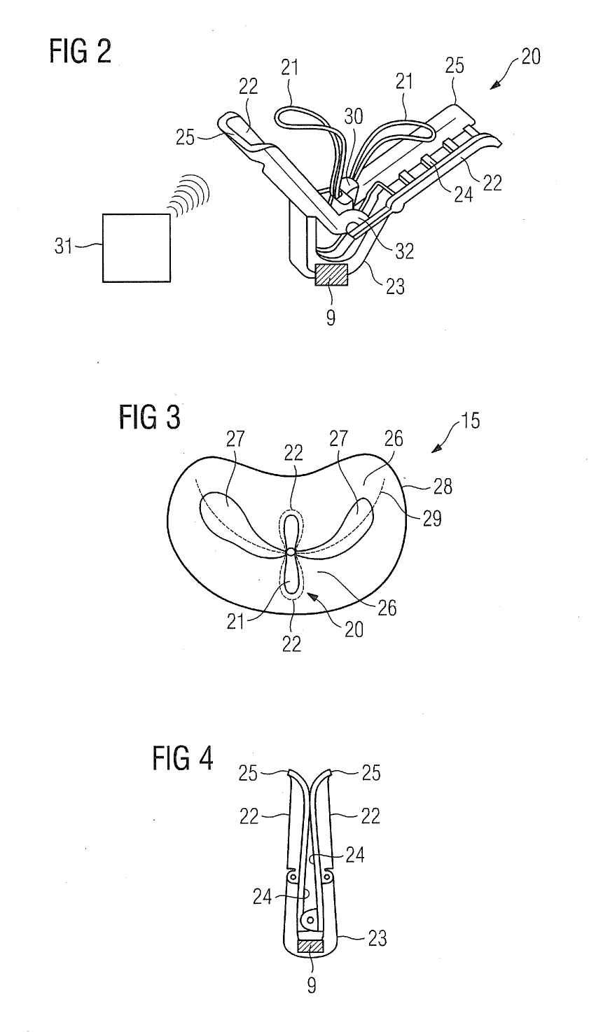

FIG. 2 shows the inventive fastening device 20 which features a position sensor 9 for measuring position information. The fastening device has two fixing arms 22, which are each connected at one end via a joint 32; the second end 25 is free. The two fixing arms 22 each have a connection side 24 and can be in the opened position (FIG. 2) or the closed position (see FIG. 4). In the closed position—as shown in FIG. 4—the fixing arms 22 are substantially parallel to one another and the connecting sides lie facing towards one another. In the opened position the free ends 25 are at a much greater distance from one another than the opposite ends connected by the joint. In the opened position (FIG. 2) the connecting sides enclose an angle of at least 5°, especially at least 20°, in relation to one another. In addition the fastening device has a drive mechanism 23, e.g. in the form of a letter U which can be pushed together to a greater or lesser extent, which supports the opening of the fixing arms 22. Two grip elements 21 are located between the foldable fixing arms. For fixing the tips of the flaps of the mitral valve are each inserted between a grip element 21 and a fixing arm 22 and the fastening device is brought into its closed position (folded together). The delivery system, e.g. with a catheter is well, can be arranged detachably on the fastening device for moving the fastening device through the body of the patient, e.g. via a docking element 30.

The position sensor 9 is arranged on the drive mechanism, but can also for example be arranged on a side of a fixing arm opposite to the connection side. The position sensor 9 involves a 5D or 6D position sensor, i.e. a position sensor from the measurements of which three space coordinates (e.g. x, y, z) can be determined for precise determination of the current location as well as also up to 3 direction angles (e.g. α, β, γ) for precise current orientation determination. The position sensor is part of a position detection system. An electromagnetic position detection system can be provided for example in this case. An electromagnetic position detection system comprises, in addition to the position sensor for example, a control and processing unit 31 to which a transmitter or field generator for generating an alternating electromagnetic field is connected. The field generator is arranged for the measurement in the vicinity of the position sensor.

The position sensor comprises for example a number of, e.g. six small coils known per se, e.g. crossed coils which can communicate wirelessly with the control and processing unit. The electromagnetic field of the field generator induces voltages in the small coils of the position sensor, which are measured by the control and processing unit and are used to determine the coordinates or the position and/or the orientation of the fastening device in a coordinate system assigned to the position detection system. Using the position detection system, the current position of the fastening device can thus be determined in each case.

The position sensor 9 and the position detection system can advantageously be used for the precise regulation of the position and orientation of the fastening device in the heart or the mitral valve. To this end the position detection system can also have a regulation device, which will for example in collaboration with a navigation device for moving the fastening device or the delivery system and in collaboration with an X-ray system for recording and displaying 3-D X-ray data, regulate a current position and orientation of the fastening device until such time as the target position and target orientation are reached.

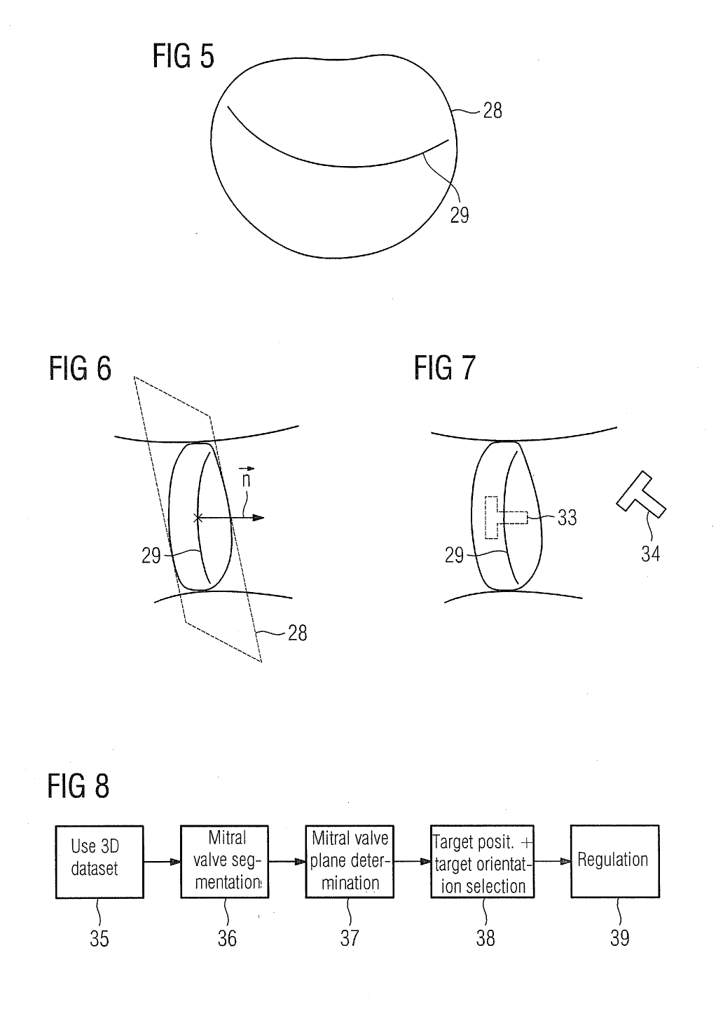

A method for delivering the fastening device into the mitral valve of a human heart can for example be carried out in the following sequence—shown in FIG. 8. Initially, in a first step (35), a 3D volume dataset of the region surrounding the mitral valve is created or a dataset already created is used. Such a volume dataset can for example be created by means of a C-arm X-ray system or a computed tomograph and subsequently displayed on a display device. From the 3D volume dataset, in a second step (36), the mitral valve is segmented either interactively by the user or automatically. Subsequently, in a third step (37), a mitral valve plane 28 of the mitral valve is determined, e.g. by three spatial points being set in the flap plane, e.g. manually by the user by clicking on them. The mitral valve plane—shown in FIG. 5 and FIG. 6—of the mitral valve can for example be defined by the Hesse normal form by \vec{r}·\vec{n}−d=0. \vec{n} designates the normal vector and \vec{r} the local vector in space in this case. The normal vector \vec{n} is at right angles to the valve plane and for example specifies an optimum direction for the delivery of the fastening device.

In a fourth step (38), which can consist of two substeps, a target position and a target orientation for the fastening device will be specified, by the user or automatically for example. This can typically be done in the form of three space coordinates (e.g. x′, y′, z′) for the target position as well as three direction angles (e.g. α′, β′, γ′) for the target orientation. In a fifth step (39), which can likewise consist of two substeps, the fastening device is for example automatically brought into the target position and the target orientation. There can also be provision for the target position to first be determined, the fastening device brought into the target position and subsequently for the target orientation to be determined and the fastening device brought into the target orientation. The navigation steps for bringing the fastening device into its target position and target orientation can be undertaken for example under visual control, by the current position and the target position being shown on a display device. With the additional arrangement of a magnetic dipole element in the fastening device or the delivery system, the longitudinal movement or angular movement can also be supported with magnetic navigation (e.g. from Stereotaxis). Shown in FIG. 7 are an unpositioned fastening device 34 and a target-positioned fastening device 33.

FIG. 3 shows in detail how an optimally positioned fastening device 20 in a faulty mitral valve 15 with an opening 27 appears. The mitral valve 15 is faulty in that a complete contact between the flaps 26 along a closure line 29 is not possible. The fastening device 20 holds the two flaps 26 of the mitral valve together in the valve plane 28, in that the flaps 26 are clamped between the fixing arms 22 and the grip elements 21 and are held together in their closed position by bringing together the fixing arms 22. This makes the opening 27 smaller, a flowback is reduced or prevented.

The position sensor can also be formed by at least one RFID transponder. The position detection system in this case also has a read device for reading out the position information of the RFID transponder, whereby the information is read out by short-range magnetic alternating fields or by high-frequency radio waves.

For a precise position determination an image coordinate system of the object under examination (patient or heart or mitral valve) and the coordinate system of the position detection system in which the coordinates of the fastening device are determined can be registered with each other for example. A registration is understood here as the determination of a coordinate transformation between the patient or an image of the patient and a coordinate system assigned to the position detection system. Registrations are known per se.

The invention can be briefly summarized in the following way: For improved delivery a fastening device is provided for connecting two flaps of a mitral valve of the human heart, having a pair of fixing arms each with a connection side, which fixing arms are connected to each other at a first end by means of a joint and each have a second free end, whereby they are able to be moved from an opened position in which the connecting sides enclose an angle of at least 5° to each other, into a closed position, in which the connecting sides are essentially parallel to one another, and featuring grip elements, which together with the fixing arms, in the closed position of the fixing arms, connect the two flaps of the mitral valve at least partly, whereby the fastening device is assigned a position detection system which is embodied to measure the spatial position and the orientation of the fastening device.

1.-11. (canceled)

12. A fastening device for connecting two flaps of a mitral valve of a human heart, comprising:
a pair of fixing arms connected to each other by a joint, wherein the pair of fixing arms have connection sides and are moved from an opened position in which the connection sides enclose an angle to each other of at least 5° into a closed position in which the connection sides are essentially in parallel to one another;
a grip element that at least partly connects the two flaps of the mitral valve in the closed position of the fixing arms; and
a position detection system that measures a spatial position and an orientation of the fastening device.

13. The fastening device as claimed in claim 12, further comprising a position sensor for determining the spatial position and the orientation of the fastening device.

14. The fastening device as claimed in claim 13, wherein the position sensor is arranged on the joint or on one of the pair of fixing arms.

15. The fastening device as claimed in claim 13, further comprising a delivery system arranged detachably on the fastening device for delivering the fastening device.

16. The fastening device as claimed in claim 15, wherein the position sensor is arranged on the delivery system.

17. The fastening device as claimed in claim 13, wherein the position sensor comprises at least one RFID.

18. The fastening device as claimed in claim 13, wherein the position sensor comprises at least one field coil.

19. The fastening device as claimed in claim 13, wherein the position detection system and the position sensor jointly determine three space coordinates as well as at least two direction angles of the fastening device.

20. The fastening device as claimed in claim 12, further comprising at least one magnetic dipole element.

21. A method for delivering a fastening device connecting two flaps of a mitral valve of a human heart, comprising:
creating a 3D volume dataset of a region surrounding the mitral valve;
segmenting the mitral valve in the 3D volume dataset;
determining a mitral valve plane of the mitral valve;
selecting a target position and a target orientation for the fastening device;
measuring a spatial position and an orientation of the fastening device by a position detection system;
regulating the measured spatial position of the fastening device to match the target position; and
regulating the measured orientation of the fastening device to match the target orientation.
